(19) United States
(12) Patent Application Publication
(10) Pub. No.: US 2020/0155470 A1
(43) Pub. Date: May 21, 2020

(54) PECTIN MICROCAPSULES, METHOD FOR THE MANUFACTURE AND USE THEREOF

(71) Applicants: UNIVERSITE DE BOURGOGNE, DlJON (FR); INSTITUT NATIONAL SUPERIEUR DES SCIENCES AGRONOMIQUES DE L'ALIMENTATION ET DE L'ENVIROMENT (AGROSUP), DIJON (FR)

(72) Inventors: Aurélie RIEU, DIJON (FR); Jean GUZZO, FONTAINE LES DIJON (FR); Ali ASSIFAOUI, DIJON (FR); Arnaud HEUMANN, DIJON (FR)

(21) Appl. No.: 16/604,591

(22) PCT Filed: Apr. 12, 2018
(86) PCT No.: PCT/FR2018/050927

§ 371 Date: Oct. 11, 2019

(30) Foreign Application Priority Data
Apr. 13, 2017 (FR) 1753248

(51) Int. Cl. A61K 9/50; A61K 35/747; A61K 35/745; A61K 36/064; A61K 35/742

(52) U.S. Cl. CPC A61K 9/5036; A61K 9/5089; A61K 35/747; A61K 45/06; A61K 36/064; A61K 35/742; A61K 35/745

(57) ABSTRACT

The invention relates to microcapsules intended to protect probiotics, comprising a shell and a core wherein the probiotics are dispersed in the form of biofilms distributed in distinct clusters, characterised in that the shell and the core are composed of pectin. The invention also relates to the method for manufacturing the capsules, the use thereof, and a formulation comprising the microcapsules according to the invention.

### TECHNICAL FIELD TO WHICH THE INVENTION RELATES

The present invention relates to the field of probiotics. In particular the present invention relates to pectin microcapsules comprising probiotics in the form of biofilm and a method for the manufacture and use thereof.

### TECHNOLOGICAL BACKGROUND

The human gastrointestinal tract comprises 10¹⁴ cohabiting microorganisms, the majority of which are situated in the colon with a concentration of around 10¹¹-10¹² bacteria/ml. By means of molecular and culture techniques that are constantly developing, at the present time more than 1000 bacterial species have been identified as resident in the human gastrointestinal tract and an individual would appear to be host to at least 160 different bacterial species. This population in the intestines, called the intestinal microbiota, plays an important role in the physiology of the host (digestion and assimilation of nutrients, protection against colonisation by pathogens and modulation of immune responses). Dysbiosis of the intestinal microbiota corresponding to an imbalance in its composition has been able to be associated with many human pathologies, such as obesity, Crohn's disease or ulcerative colitis. Ingesting probiotics may modify the composition of the intestinal microbiota and thus re-establish the balance thereof

The microorganisms most used as probiotics are bacteria belonging to the genera Lactobacillus and Bifidobacterium, natural hosts of the human intestinal microbiota. The efficacy of probiotics is specific to the strain, and each strain can contribute to the health of the host by means of different mechanisms. Among these mechanisms, modulation of the immune functions at the intestinal level is sought for probiotics with respect to the anti-inflammatory capacity thereof.

However, after ingestion, probiotics are confronted with a certain number of environmental stresses before arriving at the action site thereof, such as gastric acidity, the presence of hydrolytic enzymes or the biliary salts produced by the small intestine.

However, to be effective on the intestinal flora, probiotics must reach it in sufficient number. They must therefore in particular be capable of withstanding gastric acidity and the acidity of pancreatic juices when they pass through the stomach in order to be released alive in the intestines. In fact approximately 90% of probiotics ingested are destroyed in the face of these physical and chemical stress conditions (acid pH and high ionic strength).

In order to compensate for the bacterial mortality and to enable bacteria to better withstand the stresses encountered in the gastrointestinal tract, a first solution afforded by the prior art consists of formulating probiotics with a high bacterial load. This is because the quantity of living probiotics during ingestion must be sufficient for them to be able to persist in the gastrointestinal tract.

However, this first solution is not satisfactory since many probiotics do not withstand the stress encountered in the gastrointestinal tract before arriving at their action site. Furthermore, the production cost related to this approach is fairly great and has an effect on the final price of the probiotics.

A second solution consists of encapsulating the probiotic bacteria in a matrix, generally made from alginate, and/or adapting the probiotic bacteria to the stress conditions encountered in the gastrointestinal tract before optionally encapsulating them.

The publication by Cheow et al., 2013, BioMacromolecules, describes for example alginate or carrageenan microcapsules coated with chitosan comprising probiotics (Lactobacillus rhamnosus GG) that have been incubated in situ so as to form a biofilm. This publication shows that the encapsulation of probiotics in biofilm form improves the viability of the probiotics when they are subjected to the stress due to their lyophilisation or to a prolonged exposure to heat (60° to 70° C.). However, it has been demonstrated that alginate or carrageenan microcapsules comprising probiotics in biofilm form do not improve the viability thereof in a simulated gastric liquid compared with microcapsules comprising planktonic probiotics. This publication concludes that alginate capsules covered with chitosan represent the most appropriate system for diffusion of probiotics owing to their release profile and to their viability during storage. It also indicates that there exists a need in the prior art for providing novel capsules that incorporate additional protective materials in order to protect the probiotics to thermal and acidic exposures.

The publication by Cheow et al., 2014, Carbohydrate Polymers, which is a continuity of the publication mentioned above, describes capsules of alginate/carob seed flour and/or of alginate/xanthan gum comprising a biofilm of Lactobacillus rhamnosus GG formed in situ. These microcapsules have better resistance to stress and in particular to the acidic environment than the microcapsules described previously.

However, these microcapsules, although satisfactory, can be improved in order to afford, for example, optimum adhesion of the probiotics in the intestines.

There is also known from the prior art the publication by Nabil Aoudi and Aurélie Rieu, et al. 2014, Cellular Microbiology, which discloses in particular that bacteria, including Lactobacillus casei, in biofilm form, have anti-inflammatory properties in the intestines superior to these same bacteria cultivated in planktonic form.

Thus the conditions for culturing probiotics and the methods for formulating microcapsules/capsules are still to be defined and optimised in order in particular to improve the survival of probiotics bacteria and the functionality thereof in the intestine.

It is therefore desirable to have a novel formulation that makes it possible to provide probiotic bacteria having a survival level at the various levels of the gastrointestinal tract that is satisfactory, namely that makes it possible to produce probiotic bacteria in a viable and active form so as to be able to act in the intestine.

It is also useful to have a novel formulation that can be simple to implement and economical and can be implemented on an industrial scale.

The aim of the present invention is thus to propose a novel microcapsule at least partly avoiding the aforementioned drawbacks and making it possible in particular to improve the survival of probiotic bacteria in the gastrointestinal tract, while being simple to implement.

In particular, one aim of the invention is to provide microcapsules improving the survival of probiotic bacteria in the stomach, vectorising them into the colon and releasing them in a physiological state propitious to the implantation thereof and to the probiotic activity thereof.

### SUBJECT MATTER OF THE INVENTION

Thus the present invention relates to microcapsules intended to protect probiotics (bacteria or yeasts for example), comprising a shell and a core in which said probiotics are dispersed in the form of biofilms distributed in distinct clusters, characterised in that said shell and said core are composed of pectin.

According to the invention, “probiotics” are defined as being living microorganisms (yeasts or bacteria) exerting a beneficial action on the health of the host that ingests them by improving the balance of the intestinal flora, beyond the traditional nutritional effects. This definition was approved by the Food and Agriculture Organisation of the United Nations (FAO) and the World Health Organisation (WHO).

According to various embodiments of the invention, the following features can be used alone or in all technically possible combinations:

the biofilms resulting from the probiotics are formed in situ;

the concentration of probiotics in the microcapsules varies from 8 to 11 log CFU/g, preferably varies from 9.5 to 10.5 log CFU/g, and is typically 10 log CFU/g;

said shell and the core making up the microcapsules are made from amidated and methylated pectin having:
a degree of amidation (DA) ranging from 5% to 30%, preferably from 20% to 30%, and
a degree of esterification (DE) ranging from 5% to 50%, preferably from 20% to 30%;

the probiotics are chosen from: a probiotic bacterium such as Lactobacillus, Bifidobacterium, Enterococcus, Propionibacterium, Bacillus and Streptococcus, etc. or a yeast such as a yeast of the genus Saccharomyces cerevisiae, Saccharomyces. boulardii, etc., or one of the mixtures thereof;

said core comprises another non-probiotic active substance (namely different from a probiotic bacterium or a yeast), such as a polyphenol, a vitamin, a prebiotic, such as inulin, fructo-oligosaccharides (FOSs), etc., or one of the mixtures thereof;

the microcapsules are in dehydrated form (for example lyophilised) or frozen, so as to optimise the preserving thereof.

The present invention also relates to a probiotic formulation, characterised in that it comprises at least the microcapsules as described above.

Another subject matter of the present invention relates to a method for manufacturing microcapsules as described above, comprising the following steps:

(i) the preparation of a homogeneous mixture composed of at least: a solution or an oil/water emulsion of pectin containing a suspension of probiotics;

(ii) the encapsulation of droplets of this homogeneous mixture in a crosslinking solution, so as to form microcapsules comprising: a pectin shell and a core forming a pectin lattice, in which the probiotics are immobilised;

(iii) the culturing of the microcapsules obtained at the end of step (ii) in a culture medium, so as to form in situ in the microcapsules, biofilms distributed in distinct clusters from the immobilised probiotics;

(iv) optionally the dehydration (such as lyophilisation) or freezing of the microcapsules obtained at the end of step (iii).

According to various embodiments of the invention, the following features may be used alone or in all the technically possible combinations thereof:
the homogeneous mixture of step (i) comprises a pectin content, by mass, with respect to the total volume of the solution, ranging from 2% (m/v) to 10% (m/v), preferably from 3% (m/v) to 8% (m/v) and typically from 3.5% (m/v) to 5% (m/v);
the mixture of step (i) comprises a concentration of probiotics ranging from 10⁵ CFU/ml to 10⁹ CFU/ml, preferably from 10⁶ CFU/ml to 10^{$}CFU/ml and typically around 10⁷ CFU/ml;
the encapsulation step (ii) comprises a first substep (iia) of injection by means allowing the dropwise addition of the mixture of step (i) to the crosslinking solution stirred, so that each drop forms a microcapsule when it comes into contact with the crosslinking solution;
the encapsulation step (ii) comprises a second so-called microcapsule crosslinking step (iib) comprising the maturation of the microcapsules obtained during the injection substep (iia) over a period of at least 3 minutes, preferably ranging from 5 to 60 minutes, and in particular ranging from 10 to 25 minutes;
the crosslinking solution is composed of divalent cation, such as Ca²⁺, Zn²⁺, Ba²⁺, Fe²⁺ in chloride, sulfide or acetate form, or one of the mixtures thereof, and is in particular Ca²⁺ in chloride form;
during step (i), another non-probiotic active substance, such as polyphenol, vitamins, minerals, a prebiotic, etc., is added to the homogeneous mixture.

The present invention also relates to the use of the microcapsules as described above, or of the aforementioned probiotic formulation comprising said microcapsules, or of the microcapsules obtained according to the formation method, to protect the probiotics when passing through the stomach and to deliver them in an active form in the intestine in an animal.

“Animal” according to the invention means mammals, birds, fish, insects, etc., as well as the human being.

Another subject matter of the present invention is the microcapsules described above, or the aforementioned probiotic formulation or the microcapsules obtained according to the method described above, for use thereof as a drug.

The invention also relates to microcapsules described above, or the aforementioned probiotic formulation or the microcapsules obtained according to the method described above for use in animals, in order:
to prevent and inhibit the proliferation of a pathogen; or
to modulate the immune response; or
to influence the production of virulence factors of the pathogens in the host organism; or
to treat dysbiosis of the intestinal microbiota.

For the remainder of the description, unless it is specified otherwise, the indication of a range of values “from X to Y” or “between X and Y”, in the present invention, should be taken to include the values X and Y.

### DESCRIPTION OF THE FIGURES

The description that follows with regard to the accompanying drawings, given by way of non-limitative examples, will give a clear understanding of what the invention consists and how it can be implemented.

In the accompanying drawings:

FIG. 1 is (a) a confocal microscopy observation of a biofilm of example 1 of L. casei ATCC334 on a flat pectin film according to a kinetics of formation of a biofilm at t=24 h; and (b) is a 3D representation of this biofilm of 24 h (marking Syto9/IP); and

FIG. 2 is a confocal microscopy observation: (a) of an overall view (scale 730 μm) depicting 12 pectin microcapsules according to example 1 of the invention that were incubated in an MRS culture medium, in which there can be seen, inside each microcapsule, the various distinct clusters of biofilm comprising the probiotics L. casei ATCC334, (b) of an enlarged view (scale 150 μm) of the microcapsules of FIG. 2 (a) in which 4 microcapsules can be seen inside which there are disposed the distinct clusters of biofilm included in the pectin lattice; and (c) of an even more enlarged view (scale 5 μm) of the interior of a microcapsule of FIG. 2 (b) and showing two biofilm clusters;

FIG. 3 is a scanning electron cryomicroscopy observation: (a) of an overall view (scale 2.0 mm) of several pectin microcapsules according to example 1 of the invention that were incubated in an MRS culture medium; (b) of an enlarged view (scale (100 μm) of the interior of a microcapsule of FIG. 3 (a) in which distinct clusters of biofilm can be seen (comprising the bacteria L. casei ATCC334) distributed in the pectin lattice; (c) of an enlarged view (scale 40.0 μm) of FIG. 3 (b) in which the biofilm clusters (two in the figure) can further be seen comprising the bacteria, (d) and (e) of an even more enlarged view (scale 10.0 μm) of the interior of a biofilm cluster of FIG. 3 (c) in which the probiotics can be seen that have secreted a matrix forming a biofilm in the form of a cluster, the biofilm adhering to the wall of the pectin lattice;

FIG. 4 is a graph showing the loss of weight in a mouse over time with a DSS treatment (from day 5 to day 11): the condition “physiological water+DSS” corresponds to the mice that have received the DSS treatment and force-fed with physiological water; the condition “formulation+DSS” corresponds to the mice that have received the DSS treatment and force-fed with a formulation comprising the microcapsules according to the invention (pectin microparticles containing biofilms of L. casei probiotics);

FIG. 5 and FIG. 6 are graphs showing the quantification over time of the bacteria of the genus Lactobacillus spp (FIG. 5) and L. casei (FIG. 6) in the faeces of the group of mice that received the formulation according to said invention as mentioned in FIG. 4 (pectin microparticles containing biofilms of L. casei probiotics).

### DETAILED DESCRIPTION OF AN EXAMPLE EMBODIMENT

The following description will give a clear understanding of what the invention consists and how it can be implemented.

The Applicant has devoted itself to the development of novel microcapsules comprising probiotics able to withstand gastric acidity and acidity of the pancreatic juice when passing through the stomach in order to release the viable probiotics in the intestines, where they have in particular a beneficial action on the health of the host ingesting them.

The Applicant has discovered, unexpectedly, that microcapsules comprising a pectin shell and a core forming a pectin lattice, in which probiotics in biofilm form are immobilised, make it possible to obtain this technical effect.

This is because the Applicant has shown that such pectin microcapsules increase the survival of the probiotic bacteria or yeasts at various levels of the gastrointestinal tract and release them in viable and active form in the intestine of the host. The microcapsules according to the invention, after ingestion, therefore withstand gastric acidity, the acidity of the pancreatic juice and the hydrolytic enzymes, and furthermore become implanted in the intestine.

Thus the Applicant has developed a novel formulation of microcapsules that is simple to implement and does not require any additional protective materials in order to withstand gastric stress as is suggested in the publication of Cheow et al., 2013.

This is because, surprisingly, the simple use of pectin among all the existing polysaccharides makes it possible to form microcapsules having a pectin shell and a core composed of a pectin lattice having similar or even improved resistance to gastric stress compared with the microcapsules of the prior art, for which it is necessary to apply at least two layers of polysaccharide (alginate/chitosan) in order to protect the probiotic bacteria or yeasts.

The use of pectin is furthermore not suggested in the prior art since the three-dimensional lattice formed by the pectin in the presence of calcium ions is relatively heterogeneous with respect to the alginate lattice. This is due to the presence of an ester and amide group and the presence of branched zones in the pectin (Assifaoui et al. Soft Matter 2015). However, surprisingly, this heterogeneity of structure would appear to be responsible for the development of the probiotic bacteria and yeasts in biofilms. Thus, unlike alginate, which has a linear structure, the particular pectin gel lattice makes it possible, unexpectedly, to afford better growth of the probiotic bacteria and yeasts in this lattice.

Thus the present invention first of all relates to microcapsules intended to protect probiotics, comprising a shell and a core in which said probiotics are dispersed in the form of biofilms distributed in distinct clusters, characterised in that said shell and said core are composed of pectin.

“Biofilm” means structured communities of bacteria or yeasts enclosed in an auto-produced polymer matrix that is adherent to a living or inert surface (Costerton et al., 1999).

In particular, the biofilms issuing from probiotics are formed in situ.

It has in fact been demonstrated by the inventors that the probiotics and in particular the probiotic bacteria of the genus Lactobacillus, cultivated in a biofilm, are more resistant to stresses mimicking the conditions encountered in the gastrointestinal tract and furthermore having increased anti-inflammatory activity.

Thus putting the probiotic bacteria and yeasts in the form of a biofilm, allied with the use of pectin, makes it possible to form microcapsules having good resistance to the environmental stresses encountered between ingestion and the action site (the colon). The biofilm is moreover preserved as far as the site of delivery of the bacteria. This is because the microcapsules according to the invention are capable of releasing, in the colon, the bacteria with a biofilm phenotype, that is to say having firstly adhesion properties much superior to planktonic cells and moreover an increased probiotic activity (immunomodulation). This is because the enzyme that degrades pectin is naturally present in the colon. It is therefore a case of a targeted delivery of the bacteria in a chosen compartment of the intestine, the colon. Once naturally released from the shell and the pectin matrix, the probiotics in biofilm form can become fixed at the intestinal villosities of the colon and exert their beneficial effects on the health of their host.

Generally, the probiotic bacteria and yeasts suitable for the present invention are thus able to form a biofilm and can be chosen from: Lactobacillus, Bifidobacterium, Enterococcus, Propionibacterium, Bacillus and Streptococcus or one of the mixtures thereof.

For example, the probiotic bacteria can be chosen from: L. acidophilus, L. crispatus, L. gasseri, L. delbrueckii, L. salivarius, L. casei, L. paracasei, L. plantarum, L. rhamnosus, L. reuteri, L. brevis, L. buchneri, L. fermentum, B. adolescentis, B. angulation, B. bifidum, B. breve, B. catenulatum, B. infantis, B. lactis, B. longum, B. pseudocatenulatum, S. thermophiles or one of the mixtures thereof, and preferably the probiotic bacteria are L. casei and L. rhamnosus, or one of the mixtures thereof.

The probiotic yeasts suitable for the present invention can be chosen from: Saccharomyces cerevisiae, Saccharomyces boulardii, etc. or one of the mixtures thereof.

Advantageously the concentration of probiotics in microcapsules is very high. It varies for example from 8 to 11 log CFU/g, preferably from 9.5 to 10.5 log CFU/g, and is typically 10 log CFU/g.

In addition, according to the invention, other non-probiotic active substances, namely different from a bacterium or a yeast, may be included in the core of the microcapsules. They are in particular trapped in the pectin lattice.

These other active substances may be chosen in particular from: a polyphenol, a vitamin (thiamin (B1), riboflavin (B2), niacin (B3), pantothenic acid (B5), pyridoxine (B6), folic acid (B9) and cyanocobalamin (B12)), a mineral (magnesium, calcium, iron, etc.), a prebiotic, such as inulin, FOS, etc., or one of the mixtures thereof.

Adding these other substances such as polyphenol may make it possible for example to potentialise the effect of the biofilms.

This is because, among the food compounds, polyphenols of plant origin are highly reactive molecules which, according to in vitro and in vivo studies carried out in mice, may have a strong impact on the signalling of the intestinal cells, but also on the bacteria of the intestinal microbiota.

By way of example, it has been demonstrated for several polyphenols that they could cause an increase in the adhesion and proliferation of the probiotic bacteria such as Lactobacillus rhamnosus on the enterocytes (Parkar et al., 2008, The potential influence of fruit polyphenols on colonic microflora and human gut health, International Journal of Food Microbiology (124), p. 295-298). In addition, intestinal bacteria are known for transforming polyphenols into metabolites that may have important activities on the modulation of the intestinal homeostasis (van Duynhoven et al., 2011, Metabolic fate of polyphenols in the human superorganism, Proceedings of the National Academy of Sciences of the United States of America (108), p. 4531-4538).

It would also appear that the bioavailability of minerals, such as that of calcium, iron, zinc, manganese, copper or phosphorus, is increased in fermented dairy products compared with that of milk. Consequently the microcapsules according to the invention advantageously contain a high quantity of vitamins and minerals that will easily be assimilatable by the organism.

According to the invention, the “prebiotics” are short-chain oligosaccharides or polysaccharides consisting approximately of 2 to 20 sugar units. They escape digestion in the small intestine and are potential substrates for hydrolysis and fermentation by intestinal bacteria.

It is known that prebiotics increase the absorption of minerals (in particular calcium and magnesium) in the colon, reduce bone tissue losses, and have an effect on the immune functions. For example, the administration of inulin, oligofructose and trans-galactooligosaccharides leads to a selective increase in the faecal concentration of the bifidobacteria populations.

As mentioned above, the shell and the core of the microcapsules according to the invention are made from pectin.

In particular, the microcapsules according to the invention comprise or may consist of a shell and a core in which said probiotics are dispersed in the form of biofilms distributed in distinct clusters, characterised in that said shell and said core are composed of pectin. According to this mode, the microcapsules are solely composed of pectin in order to form the shell and the core, that is to say the microcapsules do not comprise other polysaccharides in order to form their core and shell.

Furthermore, in general, the microcapsules according to the invention do not comprise any enrobing, namely they are not covered with another protective material, such as a polysaccharide, for example chitosan.

According to the invention, the pectin is a polysaccharide of plant origin characterised by a skeleton of α-D-galacturonic acid and small quantities of α-L-rhamnose more or less branched. In particular, it comprises a concatenation of two majority structures: a homogalacturonic main chain (or “smooth zone”, called HG) and a rhamnogalacturonic chain (or “bristly zone”, called RG).

The homogalacturonans are the main chain that makes up the pectins (as a general rule representing more than 60% of the pectins). These are α-D-galacturonic acid polymers bonded in (1-4). The length of these chains may range from 70 to 100 residues of galacturonic acid in lemons, sugar beet or apples, that is to say having molar masses of around 12 to 20 kilodaltons (kDa).

The carboxylic function of α-D-galacturonic acids may be in acid form, or ionised by various cations including calcium, or esterified by methanol. Moreover, galacturonic acids may be acetylated in O-2 and/or O-3. According to these esterifications, the pectins are characterised by a degree of methylation (DM) and a degree of acetylation (DAc) that correspond to the ratio of the esterified (or respectively methylated or acetylated) galacturonic acids to the total galacturonic acids.

From a functional point of view, three categories of pectin can be distinguished:
if the degree of methylation is less than 5% (DM<5), they are pectic acids;
if the degree of methylation is less than 50% (DM<50), they are weakly methylated pectins;
if the degree of methylation is greater than 50% (DM>50), they are highly methylated (HM) pectins.

The degree of esterification of the pectins has an impact on the flexibility of the molecule: the lower the degree of esterification, the more rigid is the pectin. It also has a strong impact on their gelling properties.

In general, said shell and core making up the microcapsules are made from weakly methylated pectin that has a degree of esterification (DE=DM) ranging from 10% to 50%, preferably from 20% to 30% and typically around 28%.

The carboxylic acid function of α-D-galacturonic acids may, in the course of an industrial demethylation treatment in an ammoniacal medium, be amidated. In this case, the pectin is amidated and has a degree of amidation DA.

Preferably, said shell and the core making up the microcapsules are made from amidated pectin having a degree of amidation (DA) ranging from 3% to 30%, preferably from 20% to 30% and typically around 24%.

The degrees of amidation (DA) and esterification (DE) are determined by the titration method (Food Chemical Codex, 1981) (Food Chemical Codex. (1981). (3rd ed.). Washington, D.C.: National Academy of Sciences.

Advantageously, the microcapsules according to the invention have a mean diameter ranging from 100 μm to 5000 μm, preferably ranging from 250 μm to 1000 μm and in particular ranging from 400 μm to 800 μm.

According to one feature of the invention, the microcapsules are in dehydrated form (for example lyophilised), or frozen, so as to optimise the preservation thereof.

Then the present invention also relates to a probiotic formulation, in particular for animals, characterised in that it comprises at least the microcapsules as described above.

In particular, the probiotic formulation may be in various galenic forms preferably allowing oral taking, such as a capsule, a soft capsule, a tablet, a drink, an ampoule, powder or any other galenic form that can be ingested by a host or allowing the preparation of nutritional drinks or dishes.

By way of example, the nutritional drinks or dishes may be fermented milk, a frozen milk product, a cereal bar, a non-alcoholic drink, food supplements, a nutritional supplement, dry food and tidbits for domestic animals, etc.

As aforementioned, the present invention also relates to a method for manufacturing microcapsules as described above, comprising the following steps:

(i) the preparation of a homogeneous mixture comprising a solution or an oil/water emulsion of pectin, preferably sterilised, containing a suspension of probiotics;

(ii) the encapsulation of droplets of this mixture in a crosslinking solution, so as to form microcapsules comprising: a pectin shell and a core forming a pectin lattice, in which the probiotics are immobilised;

(iii) the culturing of the microcapsules obtained at the end of step (ii) in a culture medium so as to form in situ, in the microcapsules, biofilms distributed in distinct clusters from the immobilised probiotics;

(iv) optionally, the dehydration (such as lyophilisation) or freezing of the microcapsules obtained at the end of step (iii).

Steps (i) to (iv) will be described below.

First of all, on the one hand, a solution or an oil/water emulsion of pectin and, on the other hand, a suspension of probiotics are prepared; then the two preparations are mixed to homogenisation until a homogeneous mixture is obtained.

Naturally all the characteristics described above for the microcapsules according to the invention also apply to the method of the invention and vice versa.

In particular, the pectin included in the homogeneous mixture is preferably weakly methylated and has in particular a degree of esterification (DE=DM) ranging from 10% to 50%, preferably from 20% to 30% and typically around 28%. The pectin may also be amidated and have a degree of amidation (DA) ranging from 3% to 30%, preferably from 20% to 30% and typically around 24%.

According to a first feature of the method, the homogeneous mixture comprises a pectin content, by mass, with respect to the total volume of the mixture, ranging from 2% (m/v) to 10% (m/v), preferably from 3% (m/v) to 8% (m/v) and typically from 3.5% (m/v) to 5% (m/v).

According to a second feature of the method, the homogeneous mixture of step (i) comprises a probiotic concentration ranging from 10⁵ CFU/ml to 10⁹ CFU/ml, preferably from 10⁶ CFU/ml to 10⁸ CFU/ml and typically around 10⁷ CFU/ml.

It is possible to add to the homogeneous mixture of step (i) another non-probiotic active substance, such as polyphenol, vitamins, minerals, a prebiotic, or one of the mixtures thereof. In general, this other active substance represents by mass, with respect to the total mass of the homogeneous mixture, from 0.1% to 30%, preferably 0.1% to 20% and typically 5% to 15%.

Then the encapsulation step (ii) is carried out.

Generally, this encapsulation step (ii) comprises a first substep (iia) of injection by means allowing the dropwise addition of the homogeneous mixture of step (i) to the crosslinking solution stirred, so that each drop forms a microcapsule when it comes into contact with the crosslinking solution.

By way of example, this injection step (iia) can be carried out by means of a shower rose. In this case, the drops are divided by gravity and this in particular makes it possible to obtain microcapsules having a mean diameter ranging from 1000 to 5000 μm.

This step (iia) may also be carried out by means of an encapsulator.

In this case, the drops may, according to one embodiment, be divided by applying an electric field. The electric field applied ranging from 0.1 kV to 10 kV, preferably ranging from 2 kV to 10 kV and typically from 5 kV to 8 kV. This technique in general makes it possible to obtain microcapsules having a mean diameter ranging from 400 to 1000 μm.

According to another embodiment, the drops may be divided by vibration. This technique in general makes it possible to obtain microcapsules having a mean diameter ranging from 100 to 1000 μm.

Whatever the means allowing the dropwise application, the rate of the dropwise application varies generally from 20 μl/s to 300 μl/s, preferably varies from 40 μk/s to 100 μl/s and typically varies from 40 μl/s to 80 μl/s; while the height of injection of the homogeneous mixture of step (i) into the crosslinking solution varies from 1 cm to 15 cm, preferably varies from 2 cm to 10 cm and typically varies from 2 cm to 6 cm.

Preferably, the crosslinking solution is composed of divalent cation, such as Ca²⁺, Zn²⁺, Ba²⁺, Fe²⁺, for example in the form of chloride, sulfide or acetate, or is composed of one of the mixtures thereof. Typically, the crosslinking solution is composed of Ca²⁺ ions in the form of chloride.

In particular, the concentration of divalent ions of the crosslinking solution varies from 25 to 750 mM, preferably from 200 to 750 mM, and in particular from 500 to 750 mM.

Following this first substep (iia), the encapsulation step (ii) in particular comprises a second microcapsule crosslinking substep (iib). This second substep comprises maturing the microcapsules obtained during the injection substep (iia) for a period of at least 8 minutes, preferably ranging from 0 to 60 minutes, and in particular ranging from 10 to 25 minutes.

Advantageously, this crosslinking substep (iib) takes place at a temperature below 40° C., preferably below 30° C. and in particular ranging from 4° C. to 25° C.

Following this encapsulation step, the microcapsules thus comprise a pectin shell and a core formed by a pectin lattice in which the probiotics are immobilised.

Advantageously, the microcapsules obtained according to the method of the invention have a mean diameter ranging from 100 μm to 5000 μm, preferably ranging from 250 μm to 1000 μm and in particular ranging from 400 μm to 800 μm.

Next, prior to the culturing step (iii), the microcapsules obtained at the end of step (ii) are preferably recovered, generally by gravitational sedimentation, and then rinsed, generally with distilled water.

Then the step of culturing the microcapsules (iii) in a culture medium is performed, so as to form in situ, in the microcapsules, biofilms distributed in distinct clusters from the immobilised probiotics.

By way of example, the culture medium during this step of culturing (iii) is chosen from: MRS (deMan, Rogosa, Sharpe), AOAC (Association of Official Analytical Chemists), LB (Lysogeny Broth), TSB (Tryptic Soy Broth), TPY (Tryptone Phytone Yeast), BSM (Bifidus Selective Medium), Enterococcosel Broth (Bile Esculin Azide Broth), Nutrient Broth n° 4, CA SO Broth (Casein peptone Soybean), AC Broth (All Culture Broth), Reinforced Clostridial Medium, BHI (Brain Heart Infusion), Bifidobacterium Broth, Tomato Juice Broth, or one of the mixtures thereof, and is preferably chosen from: MRS (deMan, Rogosa, Sharpe), AOAC ((Association of Official Analytical Chemists).

Preferentially, the pH of the culture medium varies from 3 to 8, preferably from 4 to 7 and typically from 5 to 7.

The temperature of the culture medium varies for example from 20° to 40° C., preferably from 25° C. to 37° C. and generally from 25° to 30° C.

This step is in general performed for a period greater than or equal to 12 hours, preferably ranging from 12 to 72 hours and generally from 20 to 48 hours.

Finally, the freezing or dehydration of the microcapsules obtained at the end of step (iii) is optionally carried out.

One of the dehydration methods is lyophilisation. This lyophilisation step comprises for example the freezing of the microcapsules to approximately −80° C., for example in 10 ml vials, that is to say approximately 1 g of samples per vial, with a ramp of 8° C./min. Before the freezing, cryoprotectors (of the glycerol, sugar, antioxidant etc. type) may be added with the microcapsules in the vials. The samples are next lyophilised for example for 20 hours at −55° C. with a pressure of 0.05 mbar, applying 5 stages (−40, 10, 0, 20 and 30° C.). After lyophilisation, the samples are stored for example at ambient temperature.

Preferably, the freezing step is carried out by freezing the microcapsules, for example in 10 ml vials, that is to say approximately 1 g of samples per vial. Before the freezing, cryoprotectors (of the glycerol, sugar, antioxidant etc. type) may be added with the microcapsules in the vials. The freezing temperatures lie for example between −20° C. and −80° C., and the temperature drop takes place for example with a ramp of 8° C./min.

Then the present invention relates to the use of the microcapsules as described above, or of the aforementioned probiotic formulation comprising said microcapsules, or of the microcapsules obtained according to the aforementioned method, for protecting the probiotics during passage through the stomach and delivering them in an active form in the intestine in an animal.

Finally, the present invention relates to microcapsules described above, or the formation of the probiotic formulation or microcapsules obtained according to the method described above, for use thereof as a drug.

Naturally all the features described above for the microcapsules according to the invention or for the method according to the invention also apply to the aforementioned drug.

The invention also relates to microcapsules described above, or the aforementioned probiotic formulation or the microcapsules obtained according to the method described above, for use in an animal in order:
to prevent and inhibit the proliferation of pathogens; or
to modulate the immune response; or
to influence the production of virulence factors of the pathogens in the host organism; or
to treat dysbiosis of the intestinal microbiota.

Naturally all the features described above for the microcapsules according to the invention or for the method according to the invention also apply to the aforementioned uses.

### EXAMPLES

### Example 1: Preparation of Pectin Microcapsules and a Flat Pectin Film

For this test, two types of formulation were developed:

(a)—one in the form of pectin microcapsules according to the invention;

(b)—one in the form of a flat film in order best to characterise the interactions between the bacterial biofilm and the pectin gel.

A) Raw Materials

TABLE 1

| Compounds | Supplier | Characteristics |
|---|---|---|
| Methylated and amidated | Cargill, OF305C | DE: 22-28%, |
| pectin | | DA: 20-24% |
| Crosslinking medium: | Sigma Aldrich | 0.75 M |
| CaCl₂ | | |
| Probiotics tested*: | University of | strain ATCC334 |
| Lactobacillus casei | Burgundy | |
| Culture medium: MRS | Conda | pH = 5.8 |
| Culture medium: AOAC | Difco | pH 6.8 |

*These bacteria come from a culture 1% revivified for 24 h in an MRS culture medium at pH 5.8 from a 24 h culture from a cryotube in MRS pH 5.8.

B) Pectin Microcapsule Manufacturing Protocols (A)
i) preparation of a homogeneous mixture

First of all, sterile pectin in powder form is dissolved at 4% (m/v) in autoclaved ultrapure water, in a sterile atmosphere; then 10⁷ CFU/ml of bacteria is added. The whole is magnetically stirred so as to obtain a homogeneous mixture.
ii) encapsulation of droplets A Nisco Var 1 encapsulator was used (parameters: 8 kV; nozzle 0.7 mm) with a syringe driver (parameters: output 98.4 μl/h; volume 0.20 ml). The homogeneous mixture obtained above is introduced into a 20 ml syringe before being injected into the encapsulator by means of the syringe driver. The 8 kV electrical field will force the formation of microdrops, which will fall into a crosslinking bath of stirred CaCl₂, and each of these drops will form a microcapsule. The mean diameter of the wet microcapsules thus produced is around 1000 μm.

Next gelling takes place in a sterile atmosphere for 20 minutes by contact of the pectin solution with the sterile solution of 0.75 M CaCl₂.

Finally, the gel is washed three times in autoclaved ultrapure water in order to remove the excess CaCl₂.

At the end of this step, microcapsules are obtained comprising: a pectin shell and a core forming a pectin lattice, in which the probiotics are immobilised.
iii) culturing of the microcapsules The pectin microcapsules containing the immobilised bacteria (1 bath of beads=4 ml of pectin) are incubated at various times (24 h-48 h-72 h) at 28° C. in 20 ml of culture medium so as to form in situ in the microcapsules biofilms distributed in distinct clusters from the immobilised probiotics.

Two different culture media are used to allow the growth of the bacteria in a biofilm, inside the pectin microcapsules, the MRS pH=5.8 (Conda) medium or the AOAC pH 6.8 (Difco) medium.

After incubation, the pectin microcapsules containing the bacterial biofilms are washed three times in ultrapure water (15 ml).
iv) counting of the bacteria in the pectin gel The bacteria in the pectin gels are released. For this, the pectin gels (1 batch of microcapsules of 4 ml of pectin) are disintegrated using a buffer solution (50 ml) of 0.1 M sodium citrate, which releases the bacteria and suspends them in solution. The resuspended bacteria are then counted using two methods: the Colony-Forming Units method or flow cytometry.
Colony-Forming Units Method

The solution of resuspended bacteria is diluted in a cascade from 10⁻¹ to 10⁻⁵ with physiological water and 3 drops of 10 μl are deposited in the 10⁻⁵ to 10° dilutions on a gelosed MRS medium with a pH of 5.8. The Petri dish is next incubated for 48 h at 28° C. This method makes it possible to measure the bacterial cultivability.
Flow Cytometry

The solution of resuspended bacteria is first of all washed: centrifugation of 1 ml at 10,000 g and recovery of the bacterial cells in 1 ml of filtered PBS 1×. Next this bacterial suspension is diluted in order to lie between 10⁵/10⁶ bacteria/ml. Finally, the bacteria are marked with cFDA ((carboxy-Fluorescein DiAcetate) and with PI (propidium iodide). The marked bacterial solutions are then analysed by flow cytometry (BD Acuri, C6). cFDA marking makes it possible to measure the bacterial viability whereas PI marking makes it possible to measure the membrane integrity.

C) Protocols for Manufacturing a Biofilm on a Flat Pectin Film (b)
i) In order to produce flat films, the protocol consists of introducing a 4% (m/v) pectin solution into a Petri dish before covering with a dialysis membrane (Thermofisher, Snakeskin Dialysis Tubing, 10 kDa). In particular, the pectin solution is obtained by dissolving sterile pectin in powder form in autoclaved ultrapure water in a sterile atmosphere so as to obtain a pectin content of 4% (m/v).
ii) This dish is next introduced upside down (membrane downwards) for 20 minutes in a bath of CaCl₂. The calcium ions will diffuse via the dialysis membrane in the pectin solution to allow gelling of the pectin. The gel is then dried in order to form a film.
iii) To allow the growth of the probiotic bacteria in a biofilm on the surface of the pectin film, the latter is introduced for various times (24 h or 48 h) at 28° C. into the culture medium (MRS medium or AOAC medium) seeded with 10⁷ CFU/ml of bacteria. After this incubation time, the films are washed three times with ultrapure water.
iv) Counting of the bacteria released from the pectin gels: the same protocols as those described in section B)—iv were applied.

D) Results

The results of the countings obtained are presented in table 2 below and are expressed in Log (CFU) for 1 batch of capsules, which corresponds to the same quantity of pectin as 1 flat film (that is to say 4 ml of pectin at 4% (m/v)). The gels are said to be “wet” when they do not undergo any drying after gelling and the water content thereof is great.

TABLE 2

| | | Log CFU per | |
|---|---|---|---|
| | | batch of | Standard |
| | | micro- | deviation |
| | Condition | capsules | (+/−) |
| Pectin | bacteria in 24 h MRS biofilm | 10.62 | 0.03 |
| micro- | bacteria in 48 h* MRS biofilm | 10.53 | 0.28 |
| capsules | bacteria in AOAC biofilm | 9.2 | 0.03 |
| Pectin film | bacteria in 24 h MRS biofilm | 8.2 | 0.44 |

*renewal of medium at 24 h

Two methods were compared for counting the bacteria in the pectin gels: the CFU method and flow cytometry. These two methods presented closely similar results, which show good correlation between cultivability and viability of the bacteria in the pectin gels.

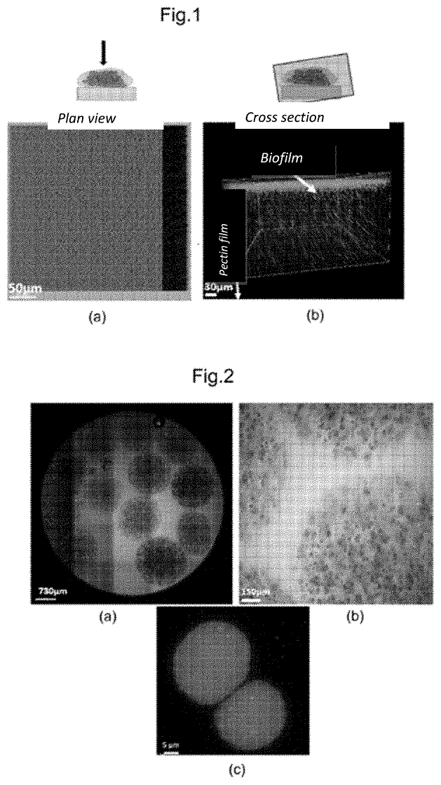

This test shows that the flat pectin films allow the growth in biofilm of the tested probiotic bacterium L. casei. In particular, as shown by FIG. 1, a uniform biofilm on the surface of the pectin film was observed by confocal microscopy.

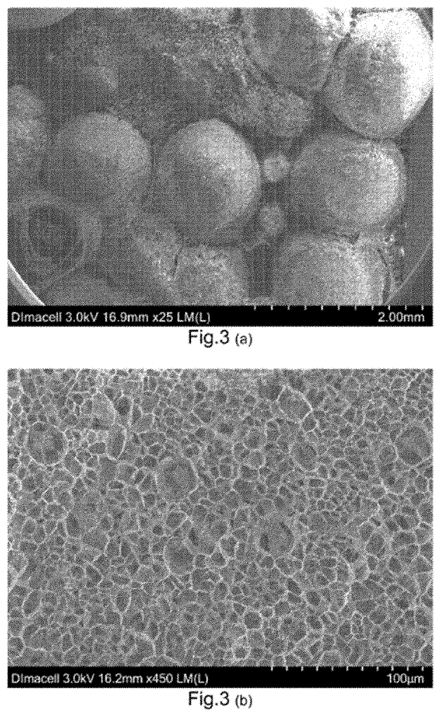

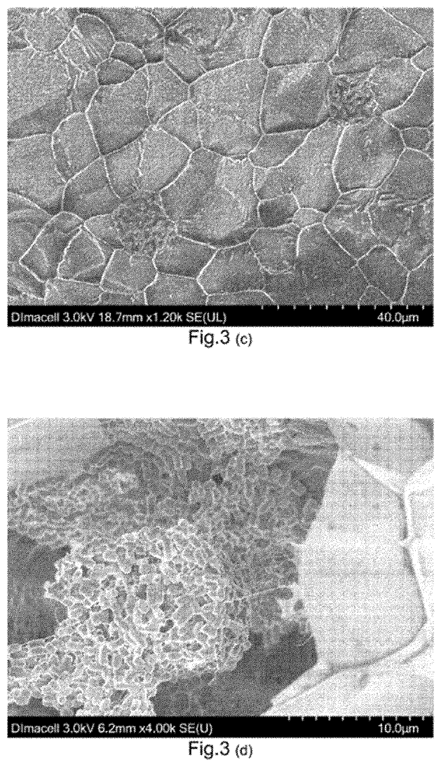

Moreover, a growth in biofilm of L. casei was also observed inside the pectin microcapsules from bacteria immobilised in the lattice of the pectin (mean increase in the initial population of 2 log).
By confocal microscopy as shown in FIG. 2, it appears that the probiotics developed in spherical microcolonies (biofilms) distributed homogeneously and separately inside the pectin microparticles cultured in the MRS medium.
In addition, by scanning electron cryomicroscopy and as shown in FIG. 3a) to FIG. 3e), it also appears that the L. casei probiotics developed in spherical microcolonies (biofilm cluster) in “niches” formed by the pectin lattice in each microcapsule. The probiotics appear to adhere to said lattice and form a veritable three-dimensional structure in the form of microcolonies (FIGS. 3(d) and 3(e)). A matrix secreted by the probiotic bacteria (exopolymer substance) and binding them to each other is also visible in FIGS. 3(d) and 3(e).

Furthermore, the Applicant also tested, under the same experimental conditions and successfully, another strain Lactobacillus rhamnosus GG in the MRS culture medium for 24 hours. This is because biofilms were formed on a flat pectin film, as well as inside the pectin microcapsules.

### Example 2: Lyophilisation of the Microcapsules of Example 1

Drying tests were carried out using lyophilisation, a method very much used in the probiotic and ferment industry, but which however does have high mortality rates.

The lyophilisation protocol is as follows:
freezing of the microcapsules at −20° C. to −80° C. in 10 ml vials, that is to say approximately 1 g of samples per vial, with a ramp of 8° C./min;
the samples are next lyophilised for 20 hours at −55° C. at a pressure of 0.05 mbar, applying 5 stages (−40, 10, 0, 20 and 30° C.);
after lyophilisation, the samples are stored at ambient temperature.

In order to check survival of the bacteria in the pectin gels after lyophilisation, a protocol for resuspending but also revivifying the bacteria was established: a batch of lyophilised pectin microcapsules (that is to say 163 mg of microcapsules +/−11 mg) is dissolved in 50 ml of a buffer solution of 0.1 M sodium citrate+MRS pH 5.8 diluted to ¼ and is incubated for 2 h at 28° C.

Countings were carried out on a gelosed culture medium in flow cytometry.

The results of the countings obtained are presented in table 3 below:

TABLE 3

| | | Log CFU per | | |
|---|---|---|---|---|
| | | batch of | Log CFU/g | Standard |
| | | micro- | of micro- | deviation |
| | Condition | capsules | capsules | (+/−) |
| Pectin | bacteria in 24 h | 9.08 | 9.86 | 0.06 |
| micro- | MRS biofilm | | | |
| capsules | bacteria in 48 h* | 7.79 | 8.44 | 0.14 |
| | MRS biofilm | | | |
| | bacteria in 24 h | 9.39 | 10.24 | 0.1 |
| | AOAC biofilm | | | |

After lyophilisation, the highest mortality was observed for planktonic bacteria, that is to say a loss of −3.02 log CFU. For the bacteria immobilised in the pectin microcapsules or for the bacteria cultivated in biofilm inside the pectin microcapsules in the MRS culture medium, the bacterial mortality after lyophilisation is similar, namely respectively 1.12 log CFU and 1.54 log CFU. The highest survival was observed for the bacteria in biofilm cultivated in AOAC medium in the pectin microcapsules, that is to say 0.4 log CFU of loss.

However, the results presented here are compatible with those found in the literature. For wet gels, it is not relevant to compare biomass values in CFU/g since, according to the gelling protocol and the polyosides used, the gels obtained may have different water contents, which will be taken into account in the weight of the gels. In the same way, according to the drying method, the water contents will be taken into account in the weight of the gels (the water contents after drying are generally not indicated). On average, in the literature, quantities of bacteria encapsulated in polyosides around 10 log CFU/g are found with drying methods optimising the survival of the bacteria. For example, for the encapsulation of Lactobacillus rhamnosus GG in biofilm in alginate/carob microcapsules, the biomass obtained is 9.7 log CFU/g (Cheow, Kiew, and Hadinoto 2014) and 9.38 log CFU/g for Bifidobacterium bifidum in alginate microspheres (Chávarri et al. 2010).

The results without optimisation of the survival of the bacteria after lyophilisation for the condition “24 h in AOAC biofilm” are 10.24 log of CFU/g and 9.86 for the condition “24 h in MRS biofilm”.

### Example 3: Test for Resistance to Acid Stress of the Microcapsules of Example 1 in Comparison with Planktonic Probiotic Bacteria

A solution mimicking the stress encountered in the stomach was used (gastric solution). This solution is composed of NaCl (0.2% m/v) and the pH is adjusted to pH=2 with 1N hydrochloric acid (according to Cook et al; 2011).

At t=0, planktonic bacteria or microcapsules (0.8 g) were introduced in a known quantity, that is to say 8.5 log CFU, in 5 ml of gastric solution at 37° C. After 2 hours of incubation, the planktonic bacteria, the bacteria in biofilm in the pectin microcapsules and the bacteria re-released in the medium were counted on MRS gelose according to the CFU method (described previously). Previously, the microcapsules were recovered and disintegrated in 10 ml of sodium citrate (0.1 M) in order to suspend the bacteria in solution.

With this protocol, the bacteria are subjected to high stress, which causes high mortality in the planktonic bacteria, around 4.6 log CFU of loss.

Contrary to this, the probiotic bacteria in biofilm in the pectin microcapsules according to the invention have a relatively low mortality compared with the planktonic bacteria, namely on average 1.15 log CFU of loss.

TABLE 4

| L. casei ATCC 334 strain | Loss in log CFU | Standard deviation |
|---|---|---|
| Planktonic bacteria | 4.6 | 0.28 |
| Microcapsules containing | 1.15 | 1.18 |
| bacteria in biofilm according | | |
| to the invention | | |

### Example 4: Test on the Adhesion Properties of the Microcapsules of Example 1 in Comparison with Planktonic Probiotic Bacteria

✓ Test 1

The adhesion of Lactobacillus casei bacteria was studied in vitro in a model of intestinal epithelial cells of the line Caco-2. The cells are cultivated in a microplate (24 wells) at a concentration of 10⁵ cells per well and maintained for 15 days in order to obtain a carpet of differentiated cells (with a brush-like border).

The Lactobacillus casei bacteria in the form of biofilm released from the pectin microcapsules according to the invention are put in contact with the epithelial cells for 1 h 30 at 37° C., at a known concentration (100 times more bacteria than epithelial cells (MOI 100), or 10 times more bacteria than epithelial cells (MOI 10)). After this incubation time, the epithelial cells are washed and the bacteria that adhered to the epithelial cells are counted on MRS geloses.

The same test is carried out with probiotic bacteria of Lactobacillus casei in planktonic form. These bacteria come from a culture revivified at 1% for 24 h in an MRS culture medium at pH 5.8 from a 24 h culture coming from a cryotube in MRS pH 5.8. These bacteria are put in contact with epithelial cells as described previously.

The degree of adhesion of the Lactobacillus casei planktonic bacteria is similar to that already described in the literature, namely 0.68% at MOI 100.

The bacteria in biofilm released from the pectin microcapsules and cultivated in MRS or AOAC medium exhibited a similar degree of adhesion, that is to say 0.7%. On the other hand, surprisingly, when these two formulations were lyophilised, the degree of adhesion increased considerably, namely to 20%.

The bacteria in biofilm in the pectin microcapsules therefore had an adhesion capacity similar to bacteria in planktonic form. The lyophilisation of the bacteria in biofilm in the pectin microcapsules increased their degree of adhesion, and first results appear to indicate that the pectin appears to play a role by promoting adhesion to the intestinal mucosa.

TABLE 5

| | MOI | Standard | MOI | Standard |
|---|---|---|---|---|
| % adhesion | 100 | deviation | 10 | deviation |
| Planktonic bacteria | 0.69 | 0.06 | 3.32 | 1.48 |
| Lyophilised planktonic | / | / | 3.69 | 0.78 |
| bacteria | | | | |
| Bacteria cultivated in MRS in | 0.75 | 0.39 | 1.08 | 0.72 |
| biofilm form released from the | | | | |
| pectin microcapsules | | | | |
| according to the invention | | | | |
| Bacteria cultivated in MRS | 21.10 | 4.69 | / | / |
| and lyophilised in biofilm form | | | | |
| released from the pectin | | | | |
| microcapsules according to | | | | |
| the invention | | | | |

✓ Test 2

In this test, the same protocol as for test 1 was used, except for the addition of pectin in solution at the step of putting the epithelial cells in contact

TABLE 6

| | | | Planktonic | Planktonic |
|---|---|---|---|---|
| | | Lyophilised | bacteria in a | bacteria in a |
| | Planktonic | planktonic | pectin gel | pectin gel |
| | bacteria | bacteria* | (4 mg) | (1.6 mg) |
| % adhesion | 3.32 | 2.23 | 44.22 | 31.21 |
| (mean) | | | | |

*according to the lyophilisation method described in example 2

This test thus demonstrates that the planktonic bacteria have a capacity for adhesion to the epithelial cells increased by the pectin. This is because the planktonic bacteria without pectin have a mean degree of adhesion of 3.32% and in contact with pectin (4 mg) this degree changes to 44.22%.

### Example 5: In Vivo Test with DSS (Dextran Sodium Sulphate)

✓ Experimental Protocol

The experimental test was carried out over 16 days.

Male C57BL mice from Charles River Laboratories were divided into two groups:

a “physiological water+DSS” group, the mice in which were force-fed daily with physiological water;

a “formulation according to the invention+DSS” group, the mice in which were force-fed daily with the formulation according to the invention containing the probiotic species Lactobacillus casei, at a dose of 10⁹ CFU/mouse/day prepared in accordance with the formulation (a) of the aforementioned example 1.

For these two groups, the mice received a DSS (Dextran Sodium Sulphate) treatment at 2% (w/v) in drinking water from day 5 to day 11 of the experiment. The DSS caused inflammation in the intestines of the mice.

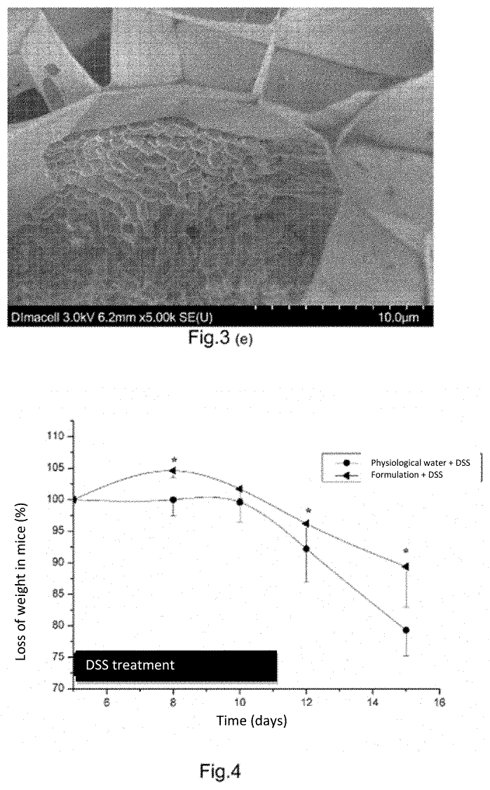

Every two days, the mice in each group were weighed in order to observe the change in weight in the mice over time (FIG. 4).

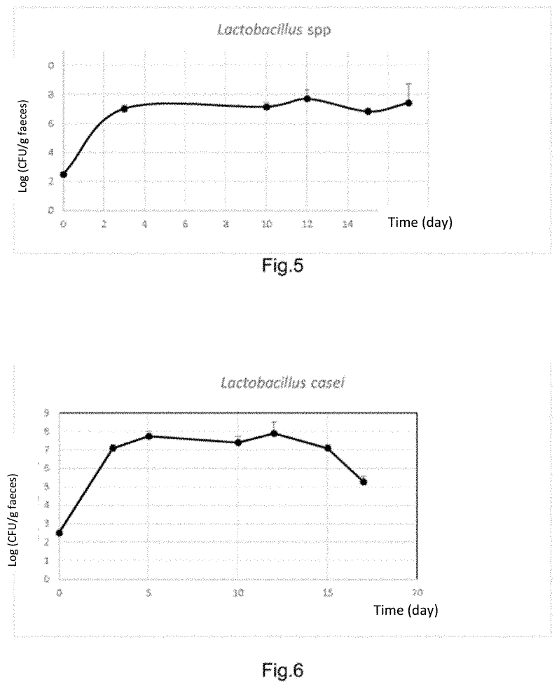

Moreover, every two days, the faeces of the mice were recovered in order to quantify the bacteria in the genus Lactobacillus on the one hand (FIG. 5) and the species L. casei on the other hand (FIG. 6). For this purpose, 140 mg of faeces from each group of mice was diluted in physiological water, and disintegrated by stirring with glass beads, giving a liquid solution (in triplicate). Finally, this solution containing the bacteria was counted in accordance with the colony-forming units method: the solution of resuspended bacteria was diluted in cascade to one tenth with physiological water and the dilutions were spread on a gelosed MRS medium at pH 6 containing 20 mg/ml of vancomycin (a medium allowing the growth of bacteria of the genus Lactobacillus) and on a gelosed M-RTLV medium at pH 6 (a medium allowing the growth of bacteria of the species L. casei). The geloses were next incubated for 48 h at 37° C. This method makes it possible to measure the bacterial cultivability.

✓ Result

This test shows first of all that the mice that received the pectin microparticles comprising the probiotic bacteria in biofilm form according to the invention have a state of health improved compared with the other mice that received physiological water. This is because the DSS caused, in the various groups of mice tested, inflammation at an intestinal level (diarrhea, loss of weight, etc.). However, the loss of weight was lesser in the group that received the formulation according to the invention, as illustrated in FIG. 4.

Next, as illustrated in FIGS. 5 and 6, this test demonstrated that the bacteria present in the biofilm contained in the pectin microcapsules according to the invention are indeed released at the intestinal level and in particular in the colon (the presence of probiotic bacteria in the faeces (8 log CFU/g).

Thus these results demonstrate that the microcapsules according to the invention allow vectorisation and release of the probiotics in viable form and make it possible to preserve the functionality of the probiotic bacteria.

1-17. (canceled)

18. Microcapsules intended to protect probiotics, comprising a shell and a core in which said probiotics are dispersed in the form of biofilms distributed in distinct clusters, wherein said shell and said core are composed of pectin.

19. Microcapsules according to claim 18, wherein the concentration of probiotics in microcapsules varies from 8 to 11 log CFU/g.

20. Microcapsules according to claim 18, wherein the concentration of probiotics in microcapsules varies from 9.5 to 10.5 log CFU/g.

21. Microcapsules according to claim 18, wherein said shell and the core making up the microcapsules are made from amidated and methylated pectin having:
a degree of amidation (DA) ranging from 5% to 30% and;
a degree of esterification (DE) ranging from 5% to 50%.

22. Microcapsules according to claim 21, wherein the degree of amidation (DA) is ranging from 5% to 30% and the degree of esterification (DE) is ranging from 20% to 30%.

23. Microcapsules according to claim 18, wherein the probiotics are chosen from: a probiotic bacterium, a yeast, or one of the mixtures thereof.

24. Microcapsules according to claim 18, wherein said core comprises another non-probiotic active substance.

25. Microcapsules according to claim 24, wherein said another non-probiotic active substance is chosen from a polyphenol, a vitamin, a prebiotic, or one of the mixtures thereof.

26. Microcapsules according to claim 18, wherein the microcapsules are provided in dehydrated or frozen form, so as to optimise preservation thereof.

27. A probiotic formulation, comprising at least the microcapsules according to claim 18.

28. A method for manufacturing microcapsules according to claim 18, comprising the following steps:
(i) the preparation of a homogeneous mixture composed of at least: a solution or an oil/water emulsion of pectin containing a suspension of probiotics;
(ii) the encapsulation of droplets of this homogeneous mixture in a crosslinking solution, so as to form microcapsules comprising: a pectin shell and a core forming a pectin lattice, in which the probiotics are immobilised;
(iii) the culturing of the microcapsules obtained at the end of step (ii) in a culture medium, so as to form in situ in the microcapsules, biofilms distributed in distinct clusters from the immobilised probiotics.

29. The method according to claim 28, wherein the homogeneous mixture of step (i) comprises a pectin content, by mass, with respect to the total volume of the solution, ranging from 2% (m/v) to 10% (m/v).

30. The method according to claim 29, wherein the homogeneous mixture of step (i) comprises a pectin content, by mass, with respect to the total volume of the solution, ranging from 3% (m/v) to 8% (m/v).

31. The method according to claim 28, wherein the mixture of step (i) comprises a concentration of probiotics ranging from 10⁵ CFU/ml to 10⁹ CFU/ml.

32. The method according to claim 28, wherein the encapsulation step (ii) comprises a first substep (iia) of injection by means allowing the dropwise addition of the mixture of step (i) to the crosslinking solution stirred, so that each drop forms a microcapsule when it comes into contact with the crosslinking solution.

33. The method according to claim 32, wherein the encapsulation step (ii) comprises a second substep (iib) of crosslinking of the microcapsules, comprising the maturation of the microcapsules obtained during the injection substep (iia) for a period of at least 3 minutes.

34. The method according to claim 28, wherein the crosslinking solution is composed of divalent cation.

35. The method according to claim 28, wherein during step (i), another non-probiotic active substance is added to the homogeneous mixture.

36. A method for protecting probiotics when passing through the stomach and delivering the probiotics in an active form in the intestine in an animal, wherein said probiotics are comprised in the microcapsules according to claim 18.

37. Microcapsules as claimed in claim 18, which is a drug.

38. Probiotic formulation as claimed in claim 27, which is a drug.
